(19) United States
(12) Patent Application Publication
(10) Pub. No.: US 2015/0366838 A1
(43) Pub. Date: Dec. 24, 2015

(54) METHOD FOR TREATING CANCER WITH A COMBINATION OF QUERCETIN AND A CHEMOTHERAPY AGENT

(71) Applicant: Thomas Christian Lines, Oberwil-Lieli (CH)

(72) Inventor: Thomas Christian Lines, Oberwil-Lieli (CH)

(21) Appl. No.: 14/744,662

(22) Filed: Jun. 19, 2015

Related U.S. Application Data
(60) Provisional application No. 62/014,488, filed on Jun. 19, 2014.

(51) Int. Cl. A61K 31/353; A61K 45/06; G01N 33/50; A61K 31/67

(52) U.S. Cl. CPC A61K 31/353; A61K 31/67; A61K 45/06; G01N 33/5011

(57) ABSTRACT

A method for treating cancer with a combination of a chemotherapy agent and a composition that includes quercetin. The composition can also include one or more of vitamin B3, vitamin C, and folic acid.

### CROSS-REFERENCE TO RELATED APPLICATIONS

The present application claims the benefit of U.S. Provisional Patent Application Ser. No. 62/014,488, which was filed on Jun. 19, 2014, by Thomas Christian Lines for a Method For Treating Cancer With a Combination of Quercetin and a Chemotherapy Agent and is hereby incorporated by reference.

### BACKGROUND

1. Field

The application relates to improving the efficacy and safety of chemotherapy agents and treating cancer with those agents.

2. Background Information

Cancer remains a leading cause of mortality, despite years of research and treatment advances. According to the US Centers for Disease Control, 575,000 individuals die from cancer each year.

Cancer is typically treated by one or a combination of modalities, including surgery, radiation, and chemotherapy. Recent advances in chemotherapy hold much promise for reducing mortality due to cancer. However, the efficacy of chemotherapy agents is often limited by their inherent toxicity, which leads to undesirable side-effects.

Side-effects of chemotherapy can include anemia, appetite changes, bleeding problems, constipation, diarrhea, fatigue, hair loss, infection, memory changes, mouth and throat changes, nausea and vomiting, nerve changes, pain, sexual and fertility changes in men and women, skin and nail changes, swelling, and urination changes.

Often, chemotherapy must be discontinued for a time or stopped completely due to the severity of the side-effects.

The need exists to improve the efficacy and reduce the side-effects of chemotherapy agents to allow for the use of lower doses of these agents while still retaining their anti-cancer activity.

### SUMMARY

To meet the existing need, the present invention features a method for treating cancer by administering to a subject in need thereof an effective amount of a chemotherapy agent together with a composition containing quercetin. The composition can be pure quercetin, e.g., 99.5% or 98.5% pure quercetin. Quercetin, as defined below, refers to both quercetin aglycon and/or quercetin derivatives. The quercetin-containing composition can also contain one or more of vitamin B3, vitamin C, and folic acid.

The details of one or more embodiments of the invention are set forth in the description below. Other features, objects, and advantages of the invention will be apparent from the description and from the claims.

### DETAILED DESCRIPTION

This invention is based, at least in part, on the unexpected finding that a composition containing quercetin, and, optionally, vitamin B3, vitamin C, or folic acid improves the efficacy of a chemotherapy agent when administered to a cancer patient.

Accordingly, the present invention features a method for treating cancer. The method includes administering to a subject suffering from cancer an effective amount of a combination of a chemotherapy agent and a composition containing quercetin and, optionally, vitamin B3, vitamin C, folic acid, or a combination thereof.

In an embodiment, a subject undergoing chemotherapy can be administered, once or periodically per day, with the composition in an amount that provides 250 mg to 5 g of quercetin (e.g., 250 mg, 500 mg, 750 mg, 1 g, 1.5 g, 2 g, 2.5 g, 3 g, 3.5 g, 4 g, 4.5 g, and 5 g). In a preferred embodiment, the composition provides 2 g of quercetin per day. In another preferred embodiment, the composition provides 5 g of quercetin per day. In an additional embodiment, the composition can be administered 2-4 times per week in a single or multiple doses over a 1, 2, 3, or 4 week period.

The improved efficacy of the chemotherapy agent mediated by quercetin is enhanced by vitamin B3, vitamin C, folic acid, or any combination thereof. For example, a combination of quercetin, vitamin B3, and vitamin C maintains quercetin levels in plasma up to five times those of quercetin alone or a combination of quercetin and vitamin B3. Further, a combination of quercetin, vitamin B3, and vitamin C results in a quercetin half-life in plasma twice as long as that of quercetin alone and about one and a half times that of a combination of quercetin and vitamin B3. Additionally, quercetin is stabilized in the presence of one or both of vitamin B3 and vitamin C.

The composition containing quercetin can also contain one or more of vitamin B3, vitamin C, and folic acid. In a particular embodiment, the composition includes quercetin, vitamin B3, vitamin C, and folic acid as the only active ingredients. A composition containing or including quercetin should also be understood to encompass pure quercetin, e.g., 99.5% pure and 98.5% pure quercetin.

In another embodiment, the cancer that can be treated is leukemia, colorectal cancer, bladder cancer, breast cancer, or kidney cancer. In yet another embodiment, the cancer is a metastatic cancer. In a specific embodiment, the cancer is metastatic bladder cancer. In yet another specific embodiment, the cancer is metastatic kidney cancer.

Cancers treatable by the above-described method include, but are not limited to acute lymphocytic leukemia, acute myeloid leukemia, adrenal cancer, adult soft tissue sarcoma, anal cancer, aplastic anemia, basal and squamous cell skin cancer, bile duct cancer, bladder cancer, bone cancer, brain/CNS tumors, breast cancer, breast cancer in man, cancer in children, cancer of unknown primary, Castleman's disease, cervical cancer, chronic lymphocytic leukemia, chronic myeloid leukemia, chronic myelomonocytic leukemia, colorectal cancer, endometrial cancer, esophagus cancer, Ewing Family of tumors, eye cancer, gallbladder cancer, gastric cancer, gastrointestinal carcinoid, gastrointestinal stromal tumor, gestational trophoblastic disease, Hodgkin's disease, Kaposi's sarcoma, kidney cancer, laryngeal and hypopharyngeal cancer, leukemia in children, liver cancer, lung cancer-non small cell, lung cancer-small cell, lung carcinoid tumor, malignant mesothelioma, melanoma skin cancer, multiple myeloma, myelodysplastic syndrome, nasal cavity and paranasal sinus cancer, nasopharyngeal cancer, neuroblastoma, non-Hodgkin's lymphoma, oral cavity and oropharangeal cancer, osteosarcoma, ovarian cancer, pancreatic cancer, penile cancer, pituitary tumors, prostate cancer, renal cell carcinoma, retinoblastoma, rhabdomyosarcoma, salivary gland cancer, skin lymphoma, small intestine cancer, stomach cancer, testicular cancer, thymus cancer, thyroid cancer, uterine sarcoma, vaginal cancer, vulvar cancer, Waldenstrom's macroglobulinemia, and Wilms' tumor.

In particular embodiments, chemotherapy agents which can be administered together with a quercetin-containing composition for treating colorectal cancer include fluorouracil, bevacizumab, irinotecan hydrochloride, capecitabine, cetuximab, oxaliplatin, leucovorin calcium, panitumumab, regorafenib, and ziv-aflibercept.

In additional embodiments, chemotherapy agents which can be administered together with a quercetin-containing composition for treating bladder cancer include cyclophosphamide, doxorubicin hydrochloride, cisplatin, gemcitabine, and a combination of gemcitabine and cisplatin.

In yet more embodiments, chemotherapy agents which can be administered together with a quercetin-containing composition for treating kidney cancer include cyclophosphamide, everolimus, aldesleukin, bevacizumab, axitinib, sorafenib tosylate, pazopanib hydrochloride, sunitinib, and temsirolimus.

Additionally, it is disclosed that a composition including quercetin can improve the efficacy of cyclophosphamide and other anti-neoplastic drugs in treating leukemia. Further, the quercetin-containing composition can be used together with low-dose cyclophosphamide to treat cancers for which low-dose cyclophosphamide alone is ineffective, including, but not limited to bladder cancer and kidney cancer.

In a particular embodiment, a cancer can be treated by co-administering a quercetin-containing composition together with one or more tyrosine kinase inhibitor. The tyrosine kinase inhibitor can be, but is not limited to, everolimus, axitinib, sorafenib tosylate, pazopanib hydrochloride, sunitinib, temsirolimus, erlotinib, and regorafenib.

Other chemotherapy agents that can be co-administered with a quercetin-containing composition include, but are not limited to altretamine, asparaginase, azacitidine, bendamustine, bleomycin, brentuximab vedotin, busulfan, cabazitaxel, carboplatin, carmustine, chlorambucil, cytarabine, dacarbazine, dactinomycin, daunorubicin, docetaxel, doxorubicin, epirubicin, eribulin mesylate, erlotinib, estramustine, etoposide, floxuridine, fludarabine, fluorouracil, gemcitabine, hydroxyurea, idarubicin, ifosfamide, irinotecan, ixabepilone, lomustine, mechlorethamine, melphalan, mercaptopurine, methotrexate, mitoxantrone, mitomycin, nelarabine, oxaliplatin, paclitaxel, pemetrexed, pentostatin, pralatrexate, temozolomide, teniposide, thioguanine, thiotepa, topotecan, vinblastine, vincristine, arsenic trioxide, clofarabine, decitabine, pegaspargase, procarbazine, romidepsin, streptozocin, and vorinostat.

The co-administration of any of the above-mentioned chemotherapy agents with a quercetin-containing composition results in (i) a reduction in tumor size, (ii) a slowing of tumor growth, or (iii) a combination of both (i) and (ii) as compared to administering the chemotherapy agent alone.

Advantageously, co-administration of a quercetin-containing composition with a chemotherapy agent reduces the side-effects associated with the chemotherapy agent. Reducing the side-effects associated with a particular chemotherapy agent allows for a higher dose to be administered. Additionally, a reduction in side-effects allows for more frequent administration of the chemotherapy agent or a prolonged period of treatment beyond that typically employed for a particular cancer.

The side-effects of a chemotherapy agent that can be reduced by co-administering it with a quercetin-containing composition include, but is not limited to, thrombosis, anemia, appetite changes, bleeding problems, constipation, diarrhea, fatigue, hair loss, infection, memory changes, mouth and throat changes, nausea and vomiting, nerve changes, pain, sexual and fertility changes in men and women, skin and nail changes, swelling, and urination changes.

In another aspect of the invention, co-administration of any of the above-mentioned chemotherapy agents with a quercetin-containing composition improves the efficacy of the chemotherapy agent with regard to the effective dose of the chemotherapy agent. More specifically, co-administration allows for the effective dose of the chemotherapy agent to be reduced by as much as 2 to 10-fold.

For example, the typical dose of the chemotherapy agent sunitinib is 50 mg per day. The dose of sunitinib can be reduced to 5-25 mg per day (e.g., 5, 10, 12.5, 15, 20, and 25 mg per day) by co-administering it with a composition including quercetin.

Similarly, the typical dose of cyclophosphamide of 3-5 mg/kg twice per week can be reduced to 0.3-2.5 mg/kg (e.g., 0.3, Q.5, 1, 1.5, 2, and 2.5 mg/kg) by co-administering it with a quercetin-containing composition.

Additionally, the typical dose of a combination of 1000 mg/m²gemcitabine and 100 mg/m² cisplatin can be reduced to 100-500 mg/m² (e.g., 100, 150, 200, 250, 300, 350, 400, 450, and 500 mg/kg) and 10-50 mg/m² (e.g., 10, 15, 20, 25, 30, 35, 40, 45, and 50 mg/kg), respectively, by co-administering these chemotherapy agents with the quercetin-containing compositions described above.

Advantageously, reducing the dose of chemotherapy agent required for treating the cancer also reduces the side-effects associated with the agent.

In an embodiment, a cancer patient who fails treatment with a particular chemotherapy agent can be successfully treated with that agent if it is co-administered with a quercetin-containing composition.

Importantly, co-administration of a composition including quercetin with a chemotherapy agent can render the agent effective against cancer cells which have become resistant to the agent over a prior course of treatment.

Co-administration of a quercetin-containing composition with a chemotherapy agent, in addition to the advantages described above, allows for the chemotherapy drug to be effective for treating a broader spectrum of cancers. For example, as mentioned above, cyclophosphamide is normally used for treating leukemia. Surprisingly, co-administration of this chemotherapy agent with a quercetin-containing composition renders the agent effective for treating stomach, kidney, and bladder cancer, including metastatic bladder and kidney cancers. Indeed, high dose cyclophosphamide is believed to cause bladder and kidney cancer in patients undergoing treatment for non-Hodgkin's lymphoma.

In a particular example, a patient bearing a tumor resulting from metastatic bladder cancer failed monotherapy with cisplatin. Subsequently, the patient was treated daily with 100 mg cyclophosphamide together with a composition containing 2 g of quercetin. A positron emission tomography (PET) scan revealed shrinkage of the tumor, and the patient reported less fatigue and an improved quality of life. Of note, this patient typically would not have been treated further after failing the monotherapy with cisplatin.

Additionally, patients having leukemia, prostate, liver, and breast cancer have been treated with daily cyclophosphamide plus quercetin, resulting in tumor shrinkage in all of these diverse cancers. In particular, breast cancer patients treated with cyclophosphamide and quercetin demonstrated an improvement in osteoporosis and osteoarthritis was also observed.

In a particular embodiment, a patient suffering from acute myeloid leukemia can be treated with a combination of doxorubicin and quercetin.

In another embodiment, breast cancer patients can be successfully treated with a combination of tamoxifen and quercetin.

The term “co-administration” can refer to any of the following: (i) combining two agents together and administering them at a single time, (ii) administering one agent and then administering a second agent a short time later (e.g., 1, 2, 5, 10, 15, 20, 30, and 45 min.; and 1, 2, 4, 6, 8, 16, and 24 hours later), (iii) administering a second agent to an individual already undergoing long-term treatment with a first agent, (iv) administering two agents simultaneously each by a different route of administration. For example, a subject suffering from cancer can be administered with a single dose of a mixture of a chemotherapy agent and a quercetin-containing composition. In another example, a subject can be administered with a chemotherapy agent via intravenous infusion and simultaneously administered orally with the quercetin-containing compositions described above. All of these co-administrations can be performed over the course of a chemotherapy cycle, e.g., 3 times per week for 3 weeks.

Additionally, a subject can be administered orally with a quercetin-containing composition for a period of time (e.g., 1, 2, 7, and 14 days) preceding the administration of a chemotherapy agent. In a particular embodiment, a cancer patient can be administered with a quercetin-containing composition each day during a standard course of chemotherapy.

In addition to the beneficial effects discussed above of treating cancer by supplementing a chemotherapy agent with quercetin, similar effects are seen in vitro for a broad spectrum of cultured cancer cells. More specifically, quercetin enhances the ability of a chemotherapy agent to kill or inhibit the growth of cultured cancer cell lines. In some instances, quercetin enhances the ability of a chemotherapy agent to induce apoptosis in cultured cancer cell lines.

In this connection, a method for identifying novel targets for a chemotherapy agent is provided. The method includes culturing a cancer cell line, contacting the cultured cancer cell line with a composition containing quercetin together with a chemotherapy agent not known to be effective against the cancer from which the cell line was derived, and measuring the growth of the cancer cell line.

This method, for example, can be used to test the effectiveness of a single chemotherapy agent against a panel of cancer cell lines to identify which cell lines can be growth-inhibited or killed by the chemotherapy agent in conjunction with quercetin. Alternatively, a library of chemotherapy agents (together with quercetin) can be tested against a single cancer cell line to determine efficacy of the agents against the particular cancer.

In this regard, provided is a method for determining the appropriate chemotherapy drug for a cancer patient. The method includes steps of isolating tumor cells from a patient, culturing the cells in vitro, contacting the cultured cells with a panel of chemotherapy agents and a quercetin-containing composition, and determining the growth of the contacted cells. The growth of the culture can be determined, e.g., by counting the number of cells and by measuring the degree of apoptosis in the culture. For example, the degree of apoptosis can be determined by immunohistochemistry. In another example, cell numbers can be determined by staining the cells with a vital dye. Cell growth and apoptosis can also be determined by flow cytometry.

The term “quercetin” refers to both quercetin aglycon and/or quercetin derivatives, e.g., quercetin-3-O-glucoside (isoquercetin), quercetin-5-O-glucoside, quercetin-7-O-glucoside, quercetin-9-O-glucoside, quercetin-3-O-rutinoside, quercetin-3-O-[α-rhamnosyl-(1→2)-α-rhamnosyl-(1→6)]-β-glucoside, quercetin-3-O-galactoside, quercetin-7-O-galactoside, quercetin-3-O-rhamnoside, quercetin-3-O-β-D-glucopyranoside (isoquercitrin), and quercetin-7-O-galactoside. After digestion, quercetin derivatives are converted to quercetin aglycon and/or other active derivatives, which are absorbed in the body. The quantity of quercetin mentioned above refers to that of quercetin aglycon or the quercetin moiety of a quercetin derivative. Quercetin can be added to the composition either in a pure form or as an ingredient in a mixture (e.g., a plant extract).

Examples of commercially available quercetin include QU995 (99.5% pure quercetin) and QU985 (98.5% pure quercetin) from Quercegen Pharmaceuticals LLC (Marlborough, Mass.).

Also commercially available from Quercegen Pharmaceuticals LLC is ISQ 995 AN (99.5% pure all-natural isoquercetin) and ISQ 995 CIT (99.5% pure isoquercitrin).

“Vitamin B3” mentioned herein includes vitamin B3 in its various forms, including niacinamide, nicotinic acid, nicotinamide, inositol hexaniacinate. Each dose of the composition can contain 20 μg-3 g vitamin B3.

“Vitamin C” mentioned herein includes vitamin C (i.e., L-ascorbic acid, D-ascorbic acid, or both) and its salts (e.g., sodium ascorbate). Each dose of the composition can contain 200 μg-3 g vitamin C.

“Folic acid” mentioned herein includes vitamin B9, folate, pteroylglutamic acid, and L-methyl folate. The amount of folate compound in a composition of this invention depends on the amounts of the other ingredients, i.e., quercetin, vitamin B3, and vitamin C. More specifically, it depends on the intended amounts of all 4 ingredients per dose or serving. It is preferred that each dose or serving contain 100-800 μg of folic acid.

The composition of this invention can be in various forms. For example, it can be a soft chew composition that includes quercetin, niacinamide, ascorbic acid, sodium ascorbate, folic acid, sugar, corn syrup, sucralose, soy lecithin, corn starch, clycerin, palm oil, xylitol, carrageenan, FD&C Yellow #6, FD&C Yellow #5, and natural and/or artificial flavors. An exemplary serving of this soft chew composition (5.15 g) includes 500 mg of quercetin, 12.9 mg of vitamin B3 (i.e., niacinamide), and 382.8 mg of vitamin C (i.e., L-ascorbic acid and sodium ascorbate). A subject can take one to eight servings (e.g., 4 servings) of this soft chew composition daily. The amounts taken can vary depending on, for example, the disorder or condition to be treated and the physical states of the subject. Another exemplary composition of this soft chew includes 5.25 wt % of quercetin, 0.25 wt % of vitamin 133, and 7.81 wt % of vitamin C (i.e., L-ascorbic acid and sodium ascorbate) plus 200 μg of folic acid per chew.

When the above-described composition is in powder form, it can be used conveniently to prepare beverage, paste, jelly, capsules, or tablets. Lactose and corn starch are commonly used as diluents for capsules and as carriers for tablets. Lubricating agents, such as magnesium stearate, are typically included in tablets.

The quercetin-containing composition of this invention can be a dietary supplement or a pharmaceutical formulation. As a dietary supplement, additional nutrients, such as minerals or amino acids may be included. The composition can also be a food product. As used herein, the term “food” broadly refers to any kinds of liquid and solid/semi-solid materials that are used for nourishing humans and animals, for sustaining normal or accelerated growth, or for maintaining stamina or alertness. Examples of human food products include, but are not limited to, tea-based beverages, juice, coffee, milk, jelly, cookies, cereals, chocolates, snack bars, herbal extracts, dairy products (e.g., ice cream, and yogurt), soy bean product (e.g., tofu), and rice products.

The terms “improving,” “enhancing,” “treating,” and “reducing” refer to the administration of an effective amount of a composition of the invention to a subject, who needs to improve one or more of the above-mentioned conditions or has one or more of the just-mentioned cancers, or a symptom or a predisposition of one of more of the cancers, with the purpose to improve one or more of these cancers, or to prevent, cure, alleviate, relieve, remedy, or ameliorate one or more of these cancers, or the symptoms or the predispositions of one or more of them. The term “administration” covers oral or parenteral delivery to a subject a composition of the invention in any suitable form, e.g., food product, beverage, tablet, capsule, suspension, and solution. The term “parenteral” refers to subcutaneous, intracutaneous, intravenous, intramuscular, intraarticular, intraarterial, intrasynovial, intrasternal, intrathecal, intralesional, and intracranial injection, as well as various infusion techniques. In this connection, a composition for administration by injection, e.g., intravenous, can include quercetin and food-grade ethanol.

An “effective amount” refers to a dose of the composition that is sufficient to provide a therapeutic benefit (e.g., reducing tumor size). Both in vivo and in vitro studies can be conducted to determine optimal administration routes and doses.

The compositions described above can be preliminarily screened for their efficacy in treating the above-described conditions in combination with chemotherapy agents by in vitro assays and then confirmed by animal experiments and clinic trials. For example, the effectiveness of the quercetin-containing compositions for enhancing the effects of a chemotherapy agent can be tested on cancer cells in culture.

Without further elaboration, it is believed that one skilled in the art can, based on the description above, utilize the present invention to its fullest extent. The specific examples below are to be construed as merely illustrative, and not limitative of the remainder of the disclosure in any way whatsoever.

### EXAMPLE 1

### Stabilization of Metastatic Bladder Cancer with Cyclophosphamide and Quercetin

In January 2010, a 64 year old underwent a cystectomy and iliac/obturator lymphadenectomy for treatment of bladder cancer. The histology report indicated that the cancer was a high grade urothelial carcinoma (stage pT3 N0). The patient was treated with 6 cycles of cisplatin plus gemcitibine between February and June of 2010. During treatment, the patient suffered from grade 2 constipation. Follow-up computed tomography (CT) and PET scans performed between July 2010 and February 2013 were negative.

A whole-body CT scan performed in February 2013 revealed an enlarged intercavoaortic lymph node (21 mm) indicating metastases. Further imaging performed between February 2013 and January 2014 indicated an increase in lymph node size to 23 mm and a change from 3.9 to 5.9 in standard uptake value (SUV) of glucose measured by PET scan.

The patient underwent 4 cycles of cisplatin plus gemcitibine between February 2014 and April 2014. Side effects included a grade 2 anemia. Follow-up CT and PET scans performed from April 2014 to December 2014 showed no improvement.

In December of 2014, the patient began daily therapy with 50 mg cyclophosphamide plus 1 g quercetin. This regimen was continued until February 2015.

The patient reported no side effects and improved quality of life. A PET scan performed in February 2015 indicated stable disease.

### EXAMPLE 2

### Treatment of Metastatic Bladder Cancer with Cyclophosphamide and Quercetin

A patient was first diagnosed with transitional cell bladder cancer (stage PT1 G1) in January 2007. The patient underwent a transurethral resection of the bladder and was treated with intravesical mitomycin C.

Follow-up between January 2007 and November 2012 was inconsistent. The patient, in November 2012, presented with blood in the urine and, in December 2012, presented with acute urinary retention.

A whole-body CT scan performed in January 2013 revealed an intravesical tumor and lymph node metastases to the obturator fossa (size of 23 mm; bilateral) and iliac (size of 15 mm; bilateral).

The patient underwent cystectomy and iliac/obturator lymphadenectomy in February of 2013. The histology report indicated that the cancer was a high grade urothelial carcinoma (stage pT3 N2). By April 2013, the cancer had spread to the interaortocaval lymph node (size of 15 mm) and the iliac lymph nodes increased in size to 33 mm.

From April 2013 to August 2013, the patient underwent 5 cycles of chemotherapy with cisplatin plus gemcitabine, and suffered from acute renal injury following the fifth cycle. The chemotherapy regimen was changed in September 2013 to carboplatin plus gemcitibine. A CT scan performed in October 2013 showed shrinkage of the involved lymph nodes, which did not shrink any further by December 2013.

A whole-body CT scan carried out in February 2014 indicated enlargement again of the involved lymph nodes (bilateral obturator fossa size of 20 mm; interaortocaval size of 20 mm; and right iliac size of 20 mm).

The patient refused standard chemotherapy in February 2014 for fear of side-effects.

The patient was treated daily with 50 mg oral cyclophosphamide plus 1 g oral quercetin from February 2014 to April 2014. A whole-body CT scan performed in April 2014 showed shrinkage of the involved lymph nodes (bilateral obturator fossa size of 15 mm; interaortocaval size of 15 mm; and right iliac size of 15 mm). The patient reported no side-effects and an improved performance status.

From April 2014 to July 2014, the patient was administered daily with 100 mg oral cyclophosphamide and 2 g oral quercetin. In July 2014, further shrinkage of the involved lymph nodes was observed by CT scan (bilateral obturator fossa size of 10 mm; interaortocaval size of 10 mm; and right iliac size of 10 mm).

The patient continued daily therapy with 100 mg oral cyclophosphamide and 2 g oral quercetin from July 2014 to February 2015 and reported no side effects.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for treating cancer, the method comprising administering to a subject in need thereof an effective amount of a combination of a chemotherapy agent and a composition containing a quercetin.

2. The method of claim 1, wherein the cancer is breast cancer, bladder cancer, kidney cancer, or colorectal cancer.

3. The method of claim 1, wherein the cancer is a metastatic cancer.

4. The method of claim 1, wherein the chemotherapy agent is a tyrosine kinase inhibitor.

5. The method of claim 4, wherein the tyrosine kinase inhibitor is everolimus, axitinib, sorafenib tosylate, pazopanib hydrochloride, sunitinib, temsirolimus, erlotinib, or regorafenib.

6. The method of claim 2, wherein the cancer is colorectal cancer and the chemotherapy agent is fluorouracil, bevacizumab, irinotecan hydrochloride, capecitabine, cetuximab, oxaliplatin, leucovorin calcium, panitumumab, regorafenib, or ziv-aflibercept.

7. The method of claim 2, wherein the cancer is bladder cancer and the chemotherapy agent is cyclophosphamide, doxorubicin hydrochloride, cisplatin, gemcitabine, or a combination of gemcitabine and cisplatin.

8. The method of claim 7, wherein the bladder cancer is metastatic and the chemotherapy agent is cyclophosphamide.

9. The method of claim 8, wherein the cyclophosphamide is administered at a daily dose of 0.3 mg/kg to 2.5 mg/kg together with 2 g of a quercetin.

10. The method of claim 9, wherein the quercetin is in the form of isoquercetin or isoquercitrin.

11. The method of claim 7, wherein the bladder cancer is metastatic and the chemotherapy agent is doxorubicin.

12. The method of claim 2, wherein the cancer is kidney cancer and the chemotherapy agent is cyclophosphamide, everolimus, aldesleukin, bevacizumab, axitinib, sorafenib tosylate, pazopanib hydrochloride, sunitinib, or temsirolimus.

13. The method of claim 12, wherein the kidney cancer is metastatic and the chemotherapy agent is sunitinib.

14. The method of claim 13, wherein the effective amount is an amount that provides 5 mg to 25 mg sunitinib and 250 mg to 5 g of a quercetin per day.

15. The method of claim 1, wherein the quercetin is selected from the group consisting of quercetin aglycone, isoquercetin, and isoquercitrin.

16. The method of claim 15, wherein the composition further includes vitamin B3, vitamin C, folic acid, or a combination thereof.

17. An in vitro method for identifying a target for a chemotherapy agent, the method comprising:
culturing a cancer cell line,
contacting the cultured cancer cell line with a chemotherapy agent known to be ineffective for inhibiting growth of the cancer cell line and a composition containing quercetin,
further culturing the contacted cultured cell line, and
measuring the growth of the cancer cell line,
wherein an inhibition of growth of the cancer cell line indicates that a cancer from which the cancer cell line was derived is a target for the chemotherapy agent.

18. The method of claim 17, wherein the composition further contains vitamin B3, vitamin C, folic acid, or a combination thereof.

19. An in vitro screening method for determining an effective chemotherapy agent for a cancer patient, the method comprising:
isolating tumor cells from a patient,
culturing the tumor cells in vitro,
contacting the cultured tumor cells with a panel of chemotherapy agents and a composition containing quercetin,
further culturing the contacted cultured tumor cells, and
measuring the growth of the tumor cells,
wherein an inhibition of growth of the tumor cells indicates that the chemotherapy agent is an effective chemotherapy agent for the cancer patient.

20. The method of claim 19, wherein the composition further contains vitamin B3, vitamin C, folic acid, or a combination thereof.
